(19) United States
(12) Patent Application Publication
(10) Pub. No.: US 2010/0256618 A1
(43) Pub. Date: Oct. 7, 2010

(54) SURGICAL INSTRUMENT

(76) Inventor: Nobuyuki Sakurazawa, Tokyo (JP)

Correspondence Address: RABIN & Berdo, PC, 1101 14TH STREET, NW, SUITE 500, WASHINGTON, DC, 20005, US

(21) Appl. No.: 12/734,750

(22) PCT Filed: Sep. 5, 2008
(86) PCT No.: PCT/JP2008/066051

§ 371 Date: May 20, 2010

(30) Foreign Application Priority Data
Nov. 22, 2007 (JP) 2007-302531

(51) Int. Cl. A61B 18/18

(52) U.S. Cl. 606/14

(57) ABSTRACT

The present invention provides a surgical instrument allowing a surgeon to identify a location of a blood vessel in tissue by hearing a blood flow sound in a thoracoscopic or laparoscopic surgery. One end of a tubular member is introduced into a body of a patient and is allowed to contact the tissue in such a manner as to block a first opening of the tubular member. Accordingly, a sound within the tissue and a sound of the tissue being in contact with the one end of the tubular member are collected by a cone member which is disposed to the first opening of the tubular member and includes a microphone disposed to the tip portion thereof. Accordingly, the surgeon can use the sense of hearing to hear the collected sound instead of the sense of touch in the thoracoscopic or laparoscopic surgery. That is, in a case where the location of the blood vessel within the tissue is to be identified, the one end of the tubular member is allowed to contact the tissue. Accordingly, the blood flow sound of the blood vessel within the tissue can be collected where the blood vessel is present in the vicinity of the contact portion. The surgeon can auscultate the collected blood flow sound, thereby identifying the location of the blood vessel. That is, even in a case where fat is embedded in the blood vessel, the location of the blood vessel can be precisely identified.

### BACKGROUND

1. Field of the Invention

The present invention relates to a surgical instrument to be introduced into a body of a patient during a thoracoscopic surgical procedure or a laparoscopic surgical procedure.

2. Description of Related Art

Conventionally, when an open abdominal surgery is performed, a surgeon palpates with his/her finger(s) to identify a location of a blood vessel within tissue inside of a body of a patient. The surgeon, however, needs to have a lot of experiences to become capable of identifying a precise location of the blood vessel. Another method for identifying the location of the blood vessel is irradiation of light of a lighting device from a backside of the tissue, that is, the light is irradiated in a direction towards the surgeon while the tissue is being provided between the light and the surgeon, so that the blood vessel within the tissue is illuminated.

Aside from the open abdominal surgery, a thoracoscopic surgery or a laparoscopic surgery, being not in need of opening the abdomen, has tended to be performed. The surgeon performs the thoracoscopic surgery or the laparoscopic surgery by creating a hole (port) to introduce a medical instrument into the abdomen or thorax and the like of the patient while watching an image provided from a camera. Accordingly, an illuminating device (e.g., Patent Document 1) or an electric scalpel to be used for the thoracoscopic surgery or the laparoscopic surgery has been developed. The illumination device includes a light emitting tube formed in a tubular shape, and is provided in the port to illuminate the inside of the body.

Patent Document 1: Japanese Unexamined Patent Application Publication No. 2006-166969

However, there is an increasing necessity of identifying the location of the blood vessel within the tissue inside of the body as the thoracoscopic or laparoscopic surgery is advanced. In the open abdominal surgery, the surgeon is allowed to easily move an organ inside the body of the patient, so that the light of the lighting device or the like is easily irradiated from the backside of the tissue. In the thoracoscopic surgery or the laparoscopic surgery, on the other hand, the surgeon is not allowed to move the organ easily, causing no space to illuminate the blood vessel within the tissue by introduction of the lighting device and the like into the body from the port. Since Patent Document 1 discloses the illuminating device to be provided in the port, the illumination device cannot be introduced into the body. Even in a case where the illuminating device as disclosed in Patent Document 1 is decreased in size, and the tissue is placed between the camera and the illuminating device, the precise location of the blood vessel embedded in the fat is difficult to be identified.

During the open abdominal surgery in general, the surgeon can identify a state of the inside of the patient's body, for example, the location of a pathological change or the blood vessel, with the sense of sight or touch. During the thoracoscopic surgery or the laparoscopic surgery, on the other hand, the surgeon can identify a state of the inside of the patient's body with only the sense of sight using the image provided from the camera so as to perform an incision and the like.

Accordingly, an inventor of the present invention has focused attention on the sense of hearing as an alternative method to the sense of touch used during the open abdominal surgery, and has found that a location of the blood vessel can be identified by a sound of the blood flowing in the blood vessel. That is, the present invention is proposed in consideration of the aforementioned situations, and provides a surgical instrument allowing a surgeon to identify a location of the blood vessel within the tissue inside the body of the patient by hearing the blood flow sound in the thoracoscopic surgery or the laparoscopic surgery.

### BRIEF SUMMARY OF THE INVENTION

According to an aspect of the present invention, a surgical instrument is introduced into a body of a patient in a thoracoscopic or laparoscopic surgery, and the surgical instrument includes: a tubular member, being hollow inside, including an opening at one end thereof; a partition member separating the opening of the tubular member into a first opening and a second opening; a cone member, disposed to the first member, formed in a taper shape towards inside of the tubular member from the first opening; a microphone disposed to a tip portion of the cone member; and a scalpel end electrode, disposed protrudably from the second opening, serving for an electric scalpel allowing coagulation and incision with electric energy.

The tubular member has the one end to be intruded into the body of the patient, and allows the one end to be in contact with the tissue in such a manner as to block the first opening of the tubular member, so that a sound within the tissue and a sound of the tissue being in contact with the one end of the tubular member are collected by the cone member which is disposed to the first opening of the tubular member and includes the microphone disposed to the tip portion thereof. Accordingly, a surgeon can use the sense of hearing to hear the collected sound instead of the sense of touch in the thoracoscopic or laparoscopic surgery.

That is, in a case where a location of the blood vessel within the tissue of the body of the patient is to be identified, the one end of the tubular member is allowed to contact the tissue. Accordingly, the blood flow sound of the blood vessel within the tissue can be collected where the blood vessel is present in the vicinity of the contact portion. The surgeon can auscultate the collected blood flow sound, thereby identifying the location of the blood vessel. Therefore, even in a case where the blood vessel is embedded in the fat, the location of the blood vessel can be precisely identified.

Moreover, since the scalpel end electrode of the electro scalpel allowing the coagulation and incision by the surgeon using the electric energy is disposed protrudably from the second opening, the surgeon can perform treatment immediately after identifying the location of the blood vessel using the auscultation.

The core member has a translucent property. The tubular member includes thereinside a light emitting unit disposed on a side opposite to the first opening through the core member. That is, the first opening of the tubular member becomes luminous by the light emitted from the light emitting unit. That is, the luminous first opening disposed to the one end of the tubular member is allowed to contact the tissue, so that the blood vessel is illuminated on the opposite side to a side being in contact with the one end of the tubular member through the tissue.

Therefore, the location of the blood vessel being not embedded in fat can be identified not only by the sense of sight but also by the sense of hearing, thereby being identified more precisely. The cone member is preferably transparent, so that the tissue can be illuminated by the light emitted from the light emitting unit without blocking the light by the cone member. Therefore, the location of the blood vessel is identified more precisely.

A membrane member having a translucent property is disposed to the first opening. The membrane member can allow the blood flow sound to be clearly heard by the surgeon. The membrane member is preferably transparent, so that the tissue can be illuminated by the light emitted from the light emitting unit without blocking the light by the membrane member. Therefore, the location of the blood vessel is identified more precisely.

Moreover, the tubular member has an oblique shaped surface including the opening. The partition member is preferably disposed to the opening of the tubular member in such a manner that the second opening is placed to a tip portion of the one end of the tubular member. Accordingly, the introduction of the tubular member into the body of the patient can be facilitated, and an area of the opening can be increased even in a case where an inner diameter of the tubular member cannot be increased. The increase in the area of the opening can allow an increase in the area of the first opening having the cone member. Accordingly, the first opening can be allowed to contact a broader area of the tissue, and the sound in the broader area can be collected.

Therefore, the location of the blood vessel in the broader area can be identified. Moreover, since the partition member is disposed to the opening of the tubular member in such a manner that the second opening is placed to the tip portion of the tubular member, the electric scalpel can be used in a state that the first opening is being in contact with the tissue and in a state that the first opening is being separated from the tissue, thereby enhancing convenience thereof.

Therefore, the surgical instrument of the present invention allows the surgeon to use the sense of hearing instead of the sense of touch in the thoracoscopic or laparoscopic surgery. In a case where the location of the blood vessel within the tissue of the body of the patient is to be identified, the one end of the tubular member is allowed to contact the tissue. Accordingly, the blood flow sound of the blood vessel within the tissue can be collected where the blood vessel is present in the vicinity of the contact portion. The surgeon can auscultate the collected blood flow sound, thereby identifying the location of the blood vessel. Therefore, even in a case where fat is embedded in the blood vessel, the location of the blood vessel can be precisely identified.

### BRIEF DESCRIPTION OF THE DRAWINGS

FIG. 1 is a schematic diagram illustrating an example of a surgical instrument according to the present invention;

FIG. 2 is a cross-sectional view illustrating of the surgical instrument taken along the line A-A′ of FIG. 1;

FIG. 3 is a schematic block diagram illustrating the surgical instrument of the present invention;

FIG. 4 is a schematic diagram illustrating a state in which a location of blood vessel is confirmed using the surgical instrument of the present invention; and

FIG. 5 is another schematic diagram illustrating a state in which a location of blood vessel is confirmed using the surgical instrument of the present invention.

### DETAILED DESCRIPTION OF PREFERRED EMBODIMENT

A surgical instrument according to preferred embodiments of the present invention is now described in detail below with reference to drawings. The present invention may, however, be embodied in many different forms and should not be construed as limited to the embodiments set forth herein. The embodiments, therefore, may be modified or varied without departing from the scope of the present invention.

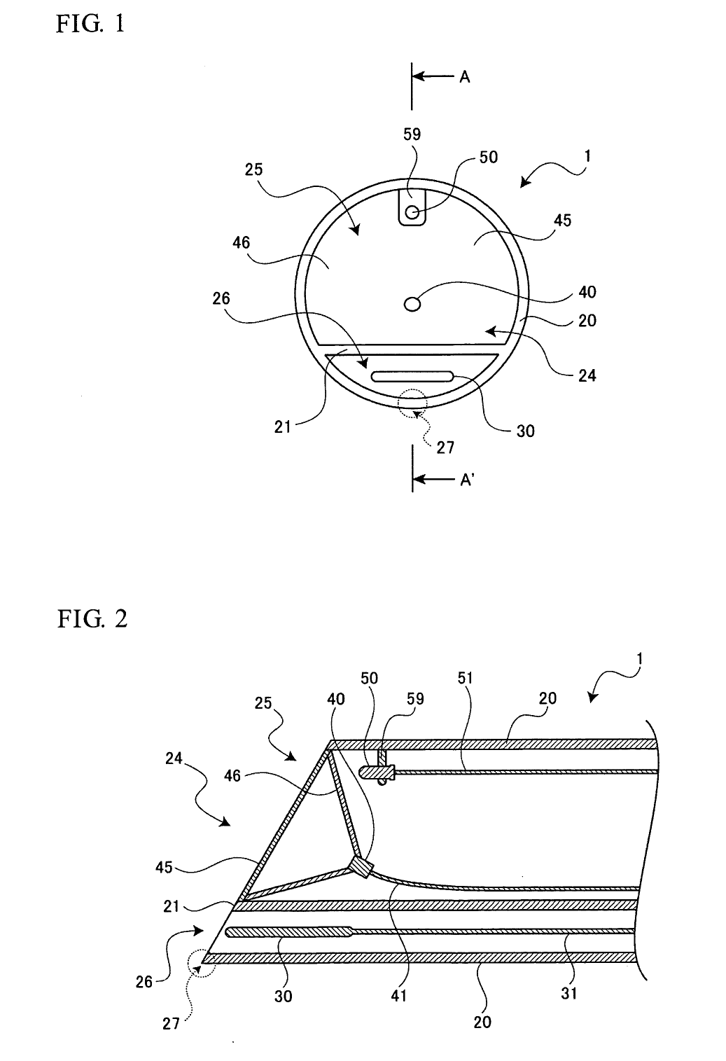

Referring to FIGS. 1 and 2, a surgical instrument 1 of the present invention is illustrated. The surgical instrument 1 includes a tubular member 20 formed in a cylindrical shape. The tubular member 20 serves as a flexible member being hollow inside, and includes an opening 24 disposed on at least one end thereof. The tubular member 20 includes the one end having a prescribed angle, for example, 45 degrees, with respect to a longitudinal direction thereof, thereby forming a surface having the opening 24. That is, the tubular member 20 includes the oblique shaped surface having the opening 24. Since the surface having the opening 24 is formed in the oblique shape, the tubular member 20 can be not only introduced into a body of a patient, but also inserted into a small space between the tissues.

The tubular member 20 has a length that is not particularly limited. However, the tubular member 20 can be any length as long as another end thereof can stay outside of the patient's body in a case of introduction into the body in the thoracoscopic surgery or the laparoscopic surgery. The tubular member 20 has an outer diameter that is not particularly limited. However, since the tubular member 20 is introduced into the body from a port having a diameter of between approximately 1 centimeter (cm) and 2 cm provided on an abdomen or the like of the body, the outer diameter thereof is preferably approximately 10 millimeters (mm). The tubular member 20 has an inner diameter determined depending on a size of the outer diameter thereof. However, the inner diameter of tubular member 20 is preferably approximately 8 mm.

The tubular member 20 can be made of any material that is not particularly limited as long as the material has flexibility. The tubular member 20 is preferably made of a material having a light shielding property. The tubular member 20 includes the one end capable of being bent or curved by an operation unit (not shown) disposed to the side of the another end thereof.

The tubular member 20 has the opening 24 including a partition member 21 formed in such a manner that a first opening 25 and a second opening 26 are formed. The partition member 21 is disposed in a position in such a manner that the inner diameter of the tubular member 20 is divided by a ratio ranged between 3:1 and 5:1. A straight line passing through a center of a circumferential cross-section of the tubular member 20 is perpendicular with respect to the partition member 21, and the tubular member 20 has a tip portion 27 placed on one end side of the straight line.

The partition member 21 can be short in length to divide only the opening 24. However, the partition member 21 can have a length that is preferably between approximately onefold and twofold with respect to a scalpel end electrode 30 (described later). Accordingly, the partition member 21 having the preferred length can reduce occurrences of making contact between the scalpel end electrode 30 and a microphone 40 (described later) or a light emitting diode (LED) 50 (described later) in a case where the scalpel end electrode 30 is moved in and out from the second opening 26. Moreover, in a case where the scalpel end electrode 30 is protruded from the second opening 26, the partition member 21 having the preferred length can guide the scalpel end electrode 30 along the tubular member 20 even in a curved state.

The first opening 24 includes a diaphragm 45, serving as a membrane member, to cover the first opening 24. The diaphragm 45 is made of a material such as resin having a translucent property allowing the light emitted from the LED 50 to be transmitted. For example, the diaphragm 45 can be made of resin such as glass epoxy resin, so that the LED 50 can emit the light to illuminate the tissue without light attenuation. The diaphragm 45 has a thickness that is not particularly limited. However, the diaphragm 45 can have a thickness to pick up a blood flow sound using the microphone 40.

The first opening 24 includes a cone member 46 formed in a tapered shape in such a manner that a tip portion of the cone member 46 is disposed inside the tubular member 20. That is, the cone member 46 is tapered from the first opening 25 towards inside the tubular member 20.

The cone member 46 is made of a material such as resin having a translucent property allowing the light emitted from the LED 50 to be transmitted, and a degree of hardness not only allowing the tapered shape thereof to be maintained but also allowing the microphone 40 to be secured inside the tubular member 20. For example, the cone member 46 can be made of transparent resin such as glass epoxy resin or acrylic resin, so that the LED 50 can emit the light to illuminate the tissue without light attenuation. The cone member 46 has a thickness that is not particularly limited, and the thickness thereof can be changed as necessary.

The microphone 40 is disposed to the tip portion of the cone member 46. The microphone 40 is disposed in such a manner as to be positioned in a space between the diaphragm 45 and the cone member 46 capable of collecting the sound. The microphone 40 includes a connection line 41 transmitting an electric signal converted from the collected sound. The connection line 41 is connected to a control unit 5 (described later) provided outside of the patient's body through inside the tubular member 20.

The LED 50, serving as a light emitting unit, is disposed opposed to the first opening 25 through the cone member 46. That is, the LED 50 is disposed to an inner side relative to the cone member 46 in the tubular member 20. The LED 50 is applied with a prescribed voltage, thereby emitting the light. The LED 50 is secured by a securing member 59 disposed inside the tubular member 20. The LED 50 includes a connection line 51 allowing the prescribed voltage to be applied to the LED 50, and the connection line 51 is connected to the control unit 5 through inside the tubular member 20.

The LED 50 has a size that is not particularly limited as long as the LED 50 can be disposed inside the tubular member 20. However, the size of the LED 50 can be arranged, for example, between approximately one-quarter and one-tenth of the inner diameter of the tubular member 20, so that the LED 50 can be disposed without contacting another member. According to the embodiment of the present invention, the LED 50 is used as an example, although the present invention is not limited thereto. For example, any substance, such as an electro-luminescent element and a light bulb, capable of emitting the light may be used as long as the size thereof is substantially similar to that of the LED 50.

The scalpel end electrode 30 of an electric scalpel is disposed inside the tubular member 20. The electric scalpel allows the coagulation and incision by the surgeon using the electric energy. The scalpel end electrode 30 is provided with a return electrode plate 39 (described later) provided outside or inside of the body in a location different from the location of the scalpel end electrode 30, so that the incision or the coagulation of the tissue is performed by the surgeon using the heat generated by the flow of high frequency electric current. The scalpel end electrode 30 is provided in such a manner as to be movable in and out from the second opening 26 through a scalpel operation unit 38 (described later) provided outside of the body in the vicinity of the another end of the tubular member 20. The scalpel end electrode 30 can be formed in a flat shape conforming to a shape of the second opening 26, although a shape of the scalpel end electrode 30 is not particularly limited.

The scalpel end electrode 30 includes an operation line 31, transmitting the operation from the scalpel operation unit 38, disposed thereto. The operation line 31 passes through inside the tubular member 20, and is connected to the control unit 5 through the scalpel operation unit 38. The operation line 31 can allow the high frequency electric current to be applied to the scalpel end electrode 30 through the control unit 5.

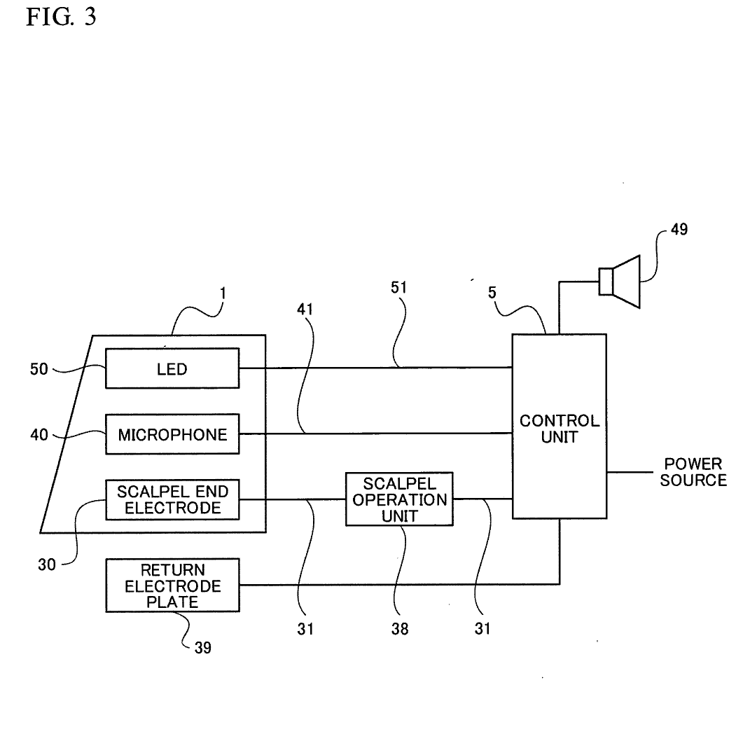

Referring to FIG. 3, the surgical instrument 1 according to the present invention is illustrated in a schematic block diagram. The surgical instrument 1 according to the present invention includes the return electrode plate 39, the scalpel operation unit 38, the control unit 5, and a speaker 49 in addition to the components described above.

The return electrode plate 39 is provided outside of the body or in the location different from the location of the tissue to be in contact with the scalpel end electrode 30 for the incision or coagulation of the tissue to be in contact with the scalpel end electrode 30. The return electrode plate 39 is connected to the control unit 5 and is connected with the scalpel end electrode 30 through the control unit 5, thereby forming the electric scalpel.

The scalpel operation unit 38 serves to operate the scalpel end electrode 30. The scalpel operation unit 38 is moved in and out from the another end of the tubular member 20, so that the scalpel end electrode 30 can be physically moved in and out from the second opening 26 through the operation line 31. However, the scalpel end electrode 30 may be moved in and out from the another end of the tubular member 20 based on the electric control. The scalpel operation unit 38 includes a switch (not shown), so that whether or not to apply the high frequency electric current to scalpel end electrode 30 is controlled using the switch. The scalpel operation unit 38 is connected to the control unit 5.

The speaker 49 receives the electric signal converted by the microphone 40, and converts the electric signal into the sound again, thereby outputting the sound collected by the microphone 40. The speaker 49 is connected to the control unit 5.

The control unit 5 is connected to a power source, and can control the light emission of the LED 50, the sound collection using the microphone 40, the sound output from the speaker 49, and the application of the high frequency electric current to be applied to the scalpel end electrode 30.

A description is now given of an example method for identifying the location of the blood vessel between the mesenteries from the aorta to the intestines using the surgical instrument 1, for example, during the laparoscopic surgical procedure.

The blood vessel within the mesentery is sandwiched by the mesenteries with fat therebetween, causing difficulty of precisely identifying the location of the blood vessel by observation using a laparoscope only. Particularly, the location of the blood vessel embedded in the fat is difficult to be identified. For example, in a case where a portion of the mesentery is incised and excised by the laparoscopic surgery, such as a case where a cancer detected in the intestine and the like and a lymph gland in the mesentery in the vicinity of the cancer are excised by the laparoscopic surgery, the precise location identification of the blood vessel within the mesenteries is extremely important. Accordingly, the present invention provides the surgical instrument 1 allowing the surgeon to precisely identify, for example, the location of the blood vessel within the mesenteries during the laparoscopic surgical procedure.

For the location identification of the blood vessel in the mesenteries, a port is first provided by piercing the abdomen of the patient to introduce the surgical instrument 1 of the present invention into the body of the patient. The surgical instrument 1 of the present invention is introduced into the body in such a manner as to inert the side having the opening 24 into the body through the port. Herein, the surgical instrument 1 is introduced into the body in such a manner that the operation unit (not shown) disposed to the another end thereof is remained outside of the body. A laparoscope (not shown) allowing the inside of the body to be displayed is introduced into the body through another port (not shown) pierced.

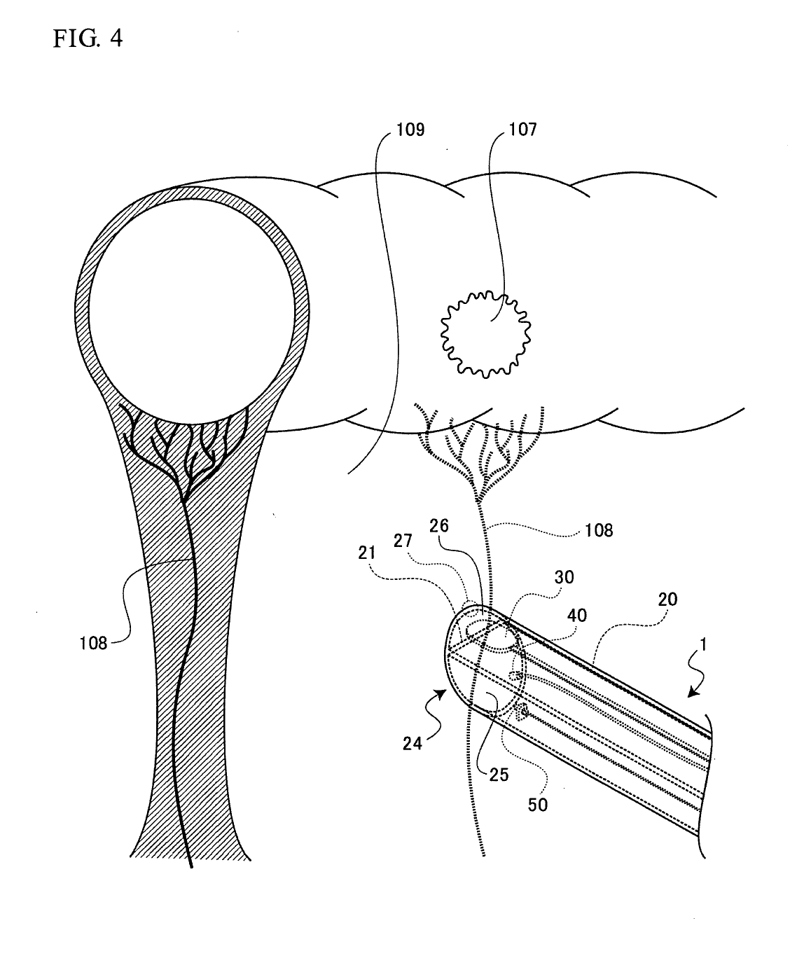

The surgical instrument 1 introduced into the body is operated using the operation unit remained outside of the body, so that the diaphragm 45 included in the first opening 25 is allowed to contact a mesentery 109 as illustrated in FIG. 4. Herein, where a blood vessel 108 is present in the vicinity of the diaphragm 45 being in contact with the mesentery 109, the microphone 40 disposed to the cone member 46 collects the sound of the blood flowing in the blood vessel 108 through the diaphragm 45. The collected blood flow sound is transmitted through the connection line 41 to the speaker 49 controlled by the control unit 5 disposed outside of the body, and is output from the speaker 49.

Since the blood flow sound is not blocked by the fat, the speaker 49 can output the blood flow sound even in a case where the blood vessel is embedded in the fat. Herein, the diaphragm 45 can block an unnecessary noise, so that the sound of the blood flow is clearly output from the speaker 49.

Where the blood vessel 108 is not present in the vicinity of the diaphragm 45 being in contact with the mesentery 109, the blood flow sound is not heard. That is, the presence or absence of the blood vessel 108 in the portion being in contact with the diaphragm 45 can be determined by whether or not the blood flow sound is heard, and the location of the blood vessel 108 can be identified by the sense of sight using the laparoscope.

The surgical instrument 1 includes the scalpel end electrode 30. The scalpel end electrode 30 is operated by the scalpel operation unit 38 and controlled by the control unit 5, so that desired tissue can be incised or coagulated. For example, in a case where a pathological change 107 in need of excision is present in the intestine as illustrated in FIG. 4, the high frequency electric current is applied with respect to the scalpel end electrode 30 in a state that the return electrode plate 39 is attached outside of the body.

Accordingly, the surgeon can incise the vicinity of the pathological change 107 using the scalpel end electrode 30 with the heat generated, thereby excising the pathological change 107. The excised pathological change 107 can be collected using forceps included in the laparoscope, and the excised portion can be coagulated with the scalpel end electrode 30 operated and controlled respectively by the operation unit 38 and the control unit 5.

Herein, since the location of the blood vessel traveling within the mesentery 109 and the intestine needs to be identified, the above described method can be applied to identify the location of the blood vessel. That is, the surgical instrument 1 according to the present invention can allow the surgeon to excise the pathological change 107 immediately after identifying the location of the blood vessel.

In a case where a scalpel end electrode is not included, an instrument having the scalpel end electrode needs to be newly introduced into a body of a patient, causing an increase in burdens on the patient due to a new port piercing and prolongation of the surgical procedure. Moreover, a tubular member having no scalpel end electrode needs to be spaced apart from the mesenteries, causing an increase in difficulty of identifying the location of the blood vessel 108. The surgical instrument 1 according to the present invention, on the other hand, can allow the surgeon to excise the pathological change 107 immediately after identifying the location of the blood vessel 108, thereby reducing risks of damaging the blood vessel 108.

The scalpel end electrode 30 allowing the incision and excision by the surgeon is disposed inside the tubular member 20, and is protruded from the second opening 26 along the tubular member 20. Accordingly, the surgeon can perform treatment on a portion covered with the opening 24 of the tubular member 20, and the operability can be enhanced compared to a case where a scalpel end electrode is operated separately from a tubular member. Moreover, a space can be used efficiently in the thorocoscopic or laparoscopic surgery performed with the limited space unlike the open abdominal surgery, thereby reducing the burdens on the patient.

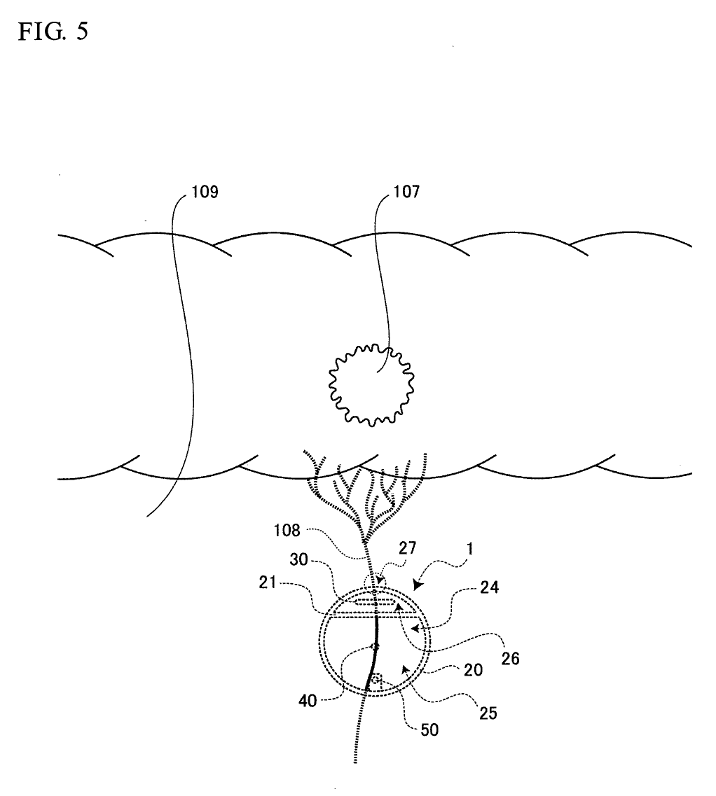

The surgical instrument 1 according to the present invention includes the LED 50. The LED 50 emits the light by application of prescribed voltage based on the control by the control unit 5. The light emitted from the LED 50 is used to illuminate the mesentery 109, so that the blood vessel within the mesentery 109 can be shown through and confirmed. In such a case, the tubular member 20 is allowed in such a manner that the laparoscope is moved to a backside of a place in which the location of the blood vessel of the mesentery 109 needs to be identified as illustrated in FIG. 5 so as to contact the first opening 25 of the tubular member 20 with the mesentery 109. Where the blood vessel 108 is present in the place in which the first opening 25 of the tubular member 20 is being in contact with the mesentery 109, the blood vessel 108 is shown through, and the location thereof can be identified by the laparoscope.

The surgical instrument 1 according to the present invention, therefore, allows the first opening 25 to be in contact with the mesentery 109, so that the diaphragm 45 is allowed to contact the mesentery 109. That is, the location of the blood vessel 108 can be confirmed not only by the sense of sight through the laparoscope, but also by the sense of hearing using the blood flow sound output through the speaker 49. Accordingly, in a hypothetical case where the blood vessel 108 is not confirmed by an image displayed using the laparoscope, the presence of the blood vessel 108 can be confirmed by the blood flow sound output from the speaker 49. Therefore, the location of the blood vessel 108 can be identified more precisely.

What is claimed is:

1. A surgical instrument to be introduced into a body of a patient in a thoracoscopic or laparoscopic surgery, the surgical instrument comprising:
a tubular member, being hollow inside, including an opening at one end thereof;
a partition member separating the opening of the tubular member into a first opening and a second opening;
a cone member, disposed to the first member, formed in a taper shape towards inside of the tubular member from the first opening;
a microphone disposed to a tip portion of the cone member; and
a scalpel end electrode, disposed protrudably from the second opening, serving for an electric scalpel allowing coagulation and incision with electric energy.

2. The surgical instrument according to claim 1, wherein the cone member has a translucent property, and
wherein the tubular member includes thereinside a light emitting unit disposed on an opposite side to the first opening through the core member.

3. The surgical instrument according to claim 2, comprising a transparent membrane member blocking the first opening.

4. The surgical instrument according to claim 1, wherein the tubular member has an oblique shaped surface including the opening; and
wherein the partition member is disposed to the opening of the tubular member in such a manner that the second opening is placed to a tip portion of the one end of the tubular member.
